(19) United States
(12) Patent Application Publication
(10) Pub. No.: US 2005/0170332 A1
(43) Pub. Date: Aug. 4, 2005

(54) Measuring method and measuring device

(76) Inventor: Atsuyoshi Shimamoto, Tokyo (JP)

Correspondence Address: KENYON & KENYON, 1 BROADWAY, NEW YORK, NY, 10004, US

(21) Appl. No.: 11/045,351

(22) Filed: Jan. 31, 2005

(30) Foreign Application Priority Data
Feb. 2, 2004 (JP) 2004-025389

(51) Int. Cl. C12Q 1/68; C12Q 1/00; G06K 9/00; C12M 1/34

(52) U.S. Cl. 435/4; 435/287.1; 382/128; 435/6

(57) ABSTRACT

The measuring device according to the present invention has a support on which cells to be observed are arranged, an imaging lens that forms an image of reflected light reflected from the cells at an image capturing position as a result of being irradiated with excitation light, and a photoelectric conversion sensor arranged at the image capturing position that converts the formed images of reflected light into images of the cells. The images of the cells captured according to first optical image capturing conditions are analyzed, second optical image forming conditions are determined corresponding to the results of that analysis, and subsequent images of the cells are captured when a predetermined amount of time has elapsed according to the second optical image capturing conditions.

### BACKGROUND OF THE INVENTION

1. Field of the Invention

The present application claims priority on Japanese Patent Application No. 2004-25389 filed on Feb. 2, 2004, the content of which is incorporated herein by reference.

The present invention relates to a fluorescence observation method for observing the fluorescence of cells and a measuring device for measuring the fluorescence of cells.

2. Description of the Related Art

Accompanying recent progress in the field of gene technology, the gene sequences of numerous organisms, including humans, have been identified. The causative relationships between gene products, such as analyzed proteins, and diseases are beginning to be elucidated, accordingly. In addition, attempts are being made to analyze various proteins and genes both comprehensively and statistically by carrying out various tests using cells. In order to carry out such tests, it is necessary to acquire predetermined information while culturing cells over an extended period of time. Consequently, there is a need to develop a device capable of culturing cells while microscopically observing them.

For example, rapid progress has been achieved in recent years in the field of technology that allows dynamic changes in cells into which genes are inserted to be observed using fluorescent light while the cells are still viable. Examples of methods used for this observation include the liposome injection method, microinjection method and gene gun method. Due to remarkable progress in the area of fluorescent proteins in the form of green fluorescent proteins (GFP) in particular, both time-based and spatial changes in organelles within viable cells as well as the behavior of various substances leading to proteins have come to be observed continuously using the fluorescent light by microscopic observation.

In the case of observing an entire sample using a microscope, the range over which a single observation can be made is mainly determined by the magnification of the objective lens of the microscope. Thus, as the magnification of the objective lens increases, the observation range ends up being limited to a very small part of the sample. Consequently, in order to acquire microscopic images of an entire sample with high resolution, the stage on which the sample is placed and the objective lens must be moved relative to each other. Partial images of the sample are thus acquired by dividing the sample into a plurality of areas, and each of those images are then synthesized into an image of the entire sample.

In order to observe a plurality of cells into which a gene has been inserted, there are cases in which fluorescent images are captured automatically at predetermined period of times. The sample, that is, the cells are placed on a single slide glass having a microarray in which an extremely large number of spots on the order of several hundred to several ten thousand spots are provided.

In this case, the types of genes inserted into each cell and the efficiency at which the genes are inserted into the cells differ for each cell. Thus, when irradiating with illumination light, the luminance of the fluorescent light emitted from the cells fluctuates with the portion of the sample that is observed. In addition, the fluorescent luminance of each cell also fluctuates with time during observation. Accordingly, there are some cells for which the fluorescent luminance exceeds the upper limit of the dynamic range of an image sensor at certain times, thereby causing saturation of the image sensor. Consequently, the observation of the fluorescence must be carried out at a dynamic range that matches the fluorescent luminance of the portion of the sample being observed. In addition, the dynamic range must be changed on a real time basis so that the image sensor does not become saturated.

A method for suitably amplifying captured fluorescent intensity is described in Japanese Patent Disclosure No. 10-305004 that uses an auto gain controller. In this method, when the output signal of an image sensor is amplified with an amplifier, the gain of the amplifier is controlled with an auto gain controller in response to the magnitude of the output signal.

### SUMMARY OF THE INVENTION

A fluorescence observation method according to a first aspect of the present invention comprises the steps of: arranging cells to be observed on a support; acquiring images of fluorescence emitted from the cells as a result of irradiating with excitation light at predetermined time intervals with a time-based image capturing system; and determining image capturing conditions such as the quantity of exposure light for capturing the fluorescent images to be subsequently acquired using the previously acquired fluorescent images to prevent saturation of fluorescent intensity.

A measuring device according to a second aspect of the present invention is provided with: a motorized stage that moves a support on which cells to be observed are arranged; a light source used to radiate excitation light onto the cells; an objective lens that converges the excitation light towards the cells; a excitation light cutoff filter that cuts out light of a predetermined wavelength from the return light reflected from the support; an imaging lens that forms an image of the fluorescent light emitted from the cells on the support at a predetermined image capturing position; an image capturing unit arranged at the image capturing position; and a processing device that incorporates fluorescent images captured by the image capturing unit and performs image processing on those images; wherein, the processing device sets the image capturing conditions of the fluorescent image to be acquired next with reference to the image processing results of the previously acquired fluorescent image.

A measuring device according to a third aspect of the present invention is the measuring device of the second aspect wherein, the image capturing unit is a solid-state image capturing unit provided with photoelectric conversion elements and in which an electronic shutter of the solid-state image capturing unit is driven based on the image capturing conditions.

A measuring device according to a fourth aspect of the present invention is the measuring device of the second aspect wherein, a mechanical shutter is arranged in front of the image capturing unit, and the mechanical shutter is driven based on the image capturing conditions.

A measuring device according to a fifth aspect of the present invention is the measuring device of the second aspect wherein, the image capturing unit is a liner-type solid-state image capturing unit in which the photoelectric conversion elements are arranged in the form of a line, and the scanning speed is changed while synchronizing the video rate of the line solid-state image capturing unit and the scanning speed of the motorized stage based on the image capturing conditions.

A measuring device according to a sixth aspect of the present invention is the measuring device of the second aspect wherein, the image capturing unit is a time delay integration type of line solid-state image capturing unit, and the number of stages of the time delay integration type of line solid-state image capturing unit is changed while synchronizing the video rate of the time delay integration type of line solid-state image capturing unit and the scanning speed of the motorized stage based on the image capturing conditions.

A measuring device according to a seventh aspect of the present invention is the measuring device of the second aspect wherein, the measuring device has a laser light source and a scanning unit that scans laser light, and the scanning speed of the laser light by the scanning unit is changed based on the image capturing conditions.

A measuring device according to an eighth aspect of the present invention is the measuring device of any of the third to seventh aspects wherein, the measuring device is additionally provided with a neutral density filter and a drive unit that drives the neutral density filter, and the neutral density filter is driven based on the image capturing conditions.

A measuring device according to a ninth aspect of the present invention is the measuring device of any of the third to seventh aspects wherein, the measuring device is additionally provided with an electrochromic light adjusting device and a control unit that controls the electrochromic light adjusting device, and the electrochromic light adjusting device is controlled based on the image capturing conditions.

A measuring device according to a tenth aspect of the present invention is the measuring device of the second aspect wherein, the support is a slide glass, and the cells are arranged in the form of a grid on the slide glass.

A measuring device according to an eleventh aspect of the present invention is the measuring device of the tenth aspect wherein, cells having a similar fluorescent intensity when a gene is expressed are arranged on the support along the scanning direction of the motorized stage, and the image capturing conditions are set for each row.

### BRIEF DESCRIPTION OF THE DRAWINGS

FIG. 1 is a block diagram showing a measuring device according to a first embodiment of the present invention.

FIG. 2 is a plan view showing an example of a motorized variable ND filter used in the measuring device of the first embodiment of the present invention.

FIG. 3 is a plan view showing the relationship between the arrangement of a sample and an image capturing area in the measuring device according to the first embodiment of the present invention.

FIG. 4 is a flow chart showing the processing of a measuring device according to the first embodiment of the present invention.

FIG. 5 is a drawing showing the relationship between the order of image capturing and the feedback of image capturing conditions for each image capturing area in the measuring device according to the first embodiment of the present invention.

FIG. 6 is a flow chart showing a fluorescent luminance analysis routine in the measuring device according to the first embodiment of the present invention.

FIG. 7 is a plan view showing another example of a motorized variable ND filter used in the measuring device according to the first embodiment of the present invention.

FIG. 8 is a block diagram showing an electrochromic light adjusting device used in a measuring device according to a second embodiment of the present invention.

FIG. 9 is a drawing showing the arrangement of the electrochromic light adjusting device in the measuring device according to the second embodiment of the present invention.

FIG. 10 is a plan view for explaining image capturing by a liner solid-state CCD camera used in a measuring device according to a third embodiment of the present invention.

FIG. 11 is a drawing showing the constitution of a TDI type of liner CCD camera used in a measuring device according to a fourth embodiment of the present invention.

FIG. 12 is a block diagram showing a measuring device according to a fifth embodiment of the present invention.

FIG. 13 is a plan view showing the arrangement of a sample on a support measured by a measuring device according to any of the first through fifth embodiments of the present invention.

### DETAILED DESCRIPTION OF THE PREFERRED EMBODIMENTS

The following provides a detailed explanation of the embodiments of the present invention with reference to the drawings.

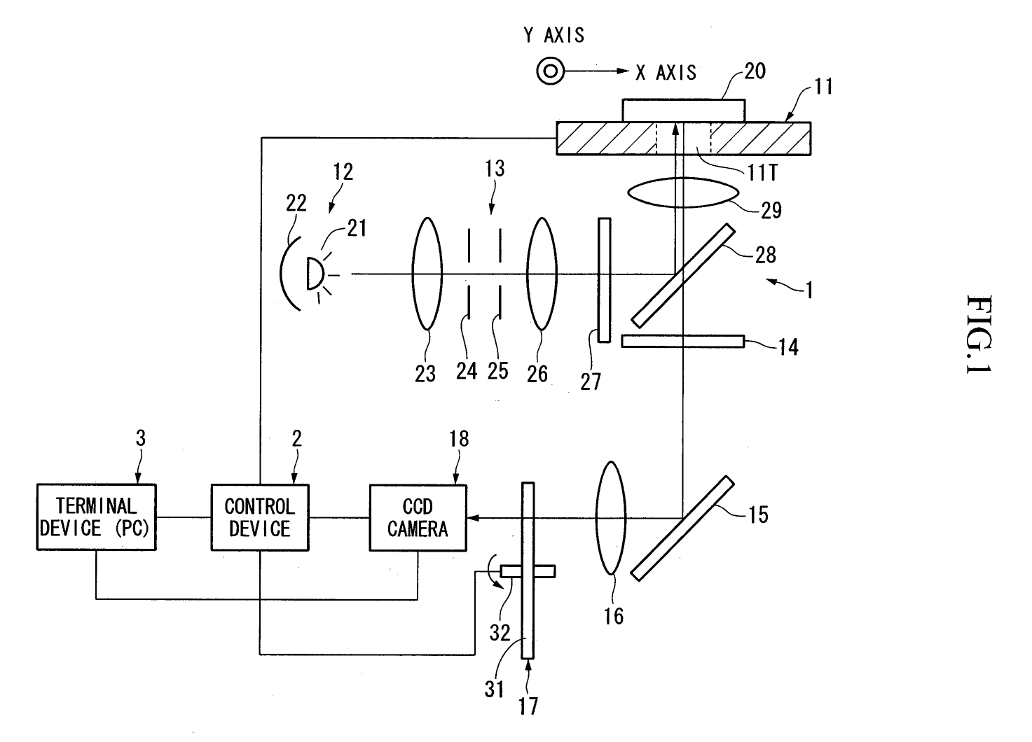

As shown in FIG. 1, a measuring device of a first embodiment of the present invention includes a microscope 1, a control device 2 connected to the microscope 1, and a terminal device 3 connected to the control device 2.

The microscope 1 has a motorized stage 11, a light source 12, an optical system 13 for fluorescent illumination, an excitation light cutoff filter 14, a mirror 15, an imaging lens 16, a motorized variable neutral density (ND) filter 17 and an image capturing system in the form of a solid-state image capturing unit 18 (hereinafter, to be referred to as a CCD (charge coupled device) camera). The constitution of microscope 1 and the number and arrangement of optical elements are not limited to the example shown in FIG. 1.

The motorized stage 11 holds a support 20 in a manner to allow two-dimensional scanning in the directions of the X axis and Y axis (perpendicular to the page) shown in the drawing. At least the portion of the motorized stage 11 on which the support 20 is placed is made from a member 11T that is transparent to excitation light and fluorescent light. The support 20 is provided to place a sample to be observed. A slide glass and so forth through which light can be transmitted is used for the support 20.

The light source 12 has a xenon lamp or other light source lamp 21 and a mirror 22. The mirror 22 is arranged behind the light source lamp 21, and efficiently collects the divergent light generated by the light source lamp 21 and directs it to the optical system 13.

The optical system 13 for fluorescent illumination includes a collector lens 23, an aperture stop 24, a field stop 25, a field lens 26, an excitation filter 27, a dichroic mirror 28 and an objective lens 29.

The collector lens 23, the aperture stop 24, the field stop 25 and the field lens 26 are arranged in that order starting from the light source 12 along the light path. The excitation filter 27 is disposed in the light path on a position which is nearer to the support 20 than the field lens 26. The excitation filter 27 allows passage of excitation light of a predetermined wavelength (e.g., 489 nm which is the central excitation wavelength of GFP). The dichroic mirror 28 is provided at an incline of about 45° relative to the optical axis below the motorized stage 11. Consequently, the excitation light can illuminate the support 20. The dichroic mirror 28 is coated with a film that reflects the excitation light but allows fluorescent light to pass through. The objective lens 29 is provided between the dichroic mirror 28 and the motorized stage 11. Fluorescent light and the excitation light are reflected by the support 20 and enter into the objective lens 29 as return light.

The excitation light cutoff filter 14 is arranged below the dichroic mirror 28. The excitation light cutoff filter 14 has the property of allowing the fluorescent light to pass through while cutting out other components of the return light. The mirror 15 is provided at an incline of about 45° with respect to the optical axis of the return light (i.e., the fluorescent light) below the excitation light cutoff filter 14. The imaging lens 16 is arranged on the optical axis of the fluorescent light reflected by the mirror 15. The imaging lens 16 focuses the image of the fluorescent light on the light receiving section (photoelectric conversion elements) of the CCD camera 18.

The CCD camera 18 is provided with an area type CCD and also has an electronic shutter. The CCD camera 18 does not capture images unless operated even if exposed to light. By controlling the operating time of the CCD camera 18 with the control device 2, therefore, it is able to function as a shutter that adjusts the amount of exposure of the fluorescent images. This type of function is referred to as an electronic shutter.

The motorized variable ND filter 17 is arranged between the imaging lens 16 and the CCD camera 18. The motorized variable ND filter 17 is composed of a disk-shaped ND filter 31, a rotation axis 32 that passes through the center of the ND filter 31, and a drive unit (not shown) that rotates the rotation axis 32. As an example of the drive unit, a motor coupled to the rotation axis 32 is employed.

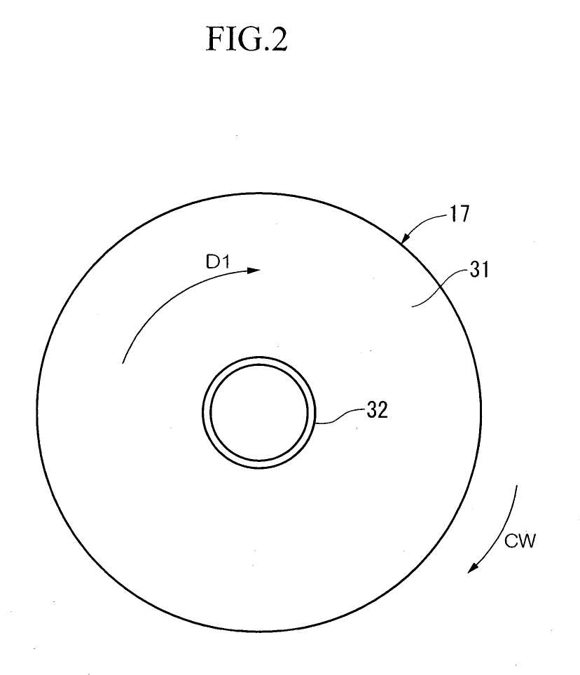

An example of the motorized variable ND filter 17 is shown in FIG. 2. In FIG. 2, the rotation axis 32 passes perpendicularly through the center of the ND filter 31. The ND filter 31 has variable density along the circumferential direction indicated with arrow D1, and its transmittance changes continuously. Thus, when the rotation axis 32 is rotated in the clockwise (CW) direction by the control device 2, the ND filter 31 rotates accompanying this rotation and the transmittance of light that passes through it increases or decreases, accordingly.

Referring to FIG. 1 again, the CCD camera 18 is provided with a light receiving section in which photoelectric conversion elements are arranged that receive fluorescent light and generate an electrical signal.

The control device 2 is connected to the terminal device 3, the motorized stage 11, the light source 12, the motorized variable ND filter 17 and the CCD camera 18. The control device 2 includes a central processing unit (CPU), a memory and so forth to control the operations of the terminal device 3, the motorized stage 11, the light source 12, the motorized variable ND filter 17 and the CCD camera 18 based on a predetermined program. In particular, it adjusts the quantity of light that enters the CCD camera 18 according to a fluorescent luminance analysis routine (FIG. 6) to be described later based on the fluorescent images produced with the terminal device 3.

The terminal device 3 is connected to the control device 2 and the CCD camera 18. A personal computer, for example, can be used for the terminal device 3. The terminal device 3 incorporates and stores the fluorescent images of the cells captured with the CCD camera 18. Moreover, it displays the fluorescent images on a screen and outputs the fluorescent images to a paper medium or recording medium. In addition, it accepts operations by an observer and transfers necessary information to the control device 2.

Next, an explanation is provided of the operation of the first embodiment.

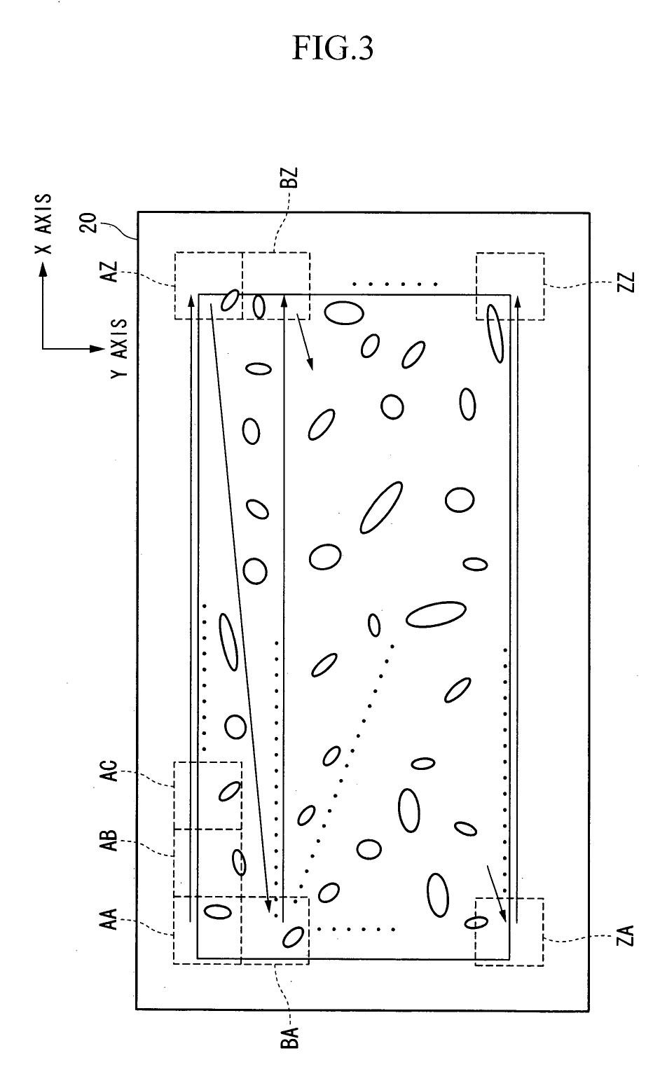

First, a sample is held on the support 20 prior to image capturing. FIG. 3 shows an example of the arrangement of the sample on the support 20. Tissue and so forth to be observed is dispersed in a predetermined region of the sample. In this type of sample, the regions in which fluorescent images are to be acquired, namely the image capturing areas, are those regions in which the objects to be observed are present, and are indicated with reference symbols AA to ZZ in FIG. 3. For example, an image capturing area AC is located at the third area in the direction of the X axis and the first area in the direction of the Y axis. Furthermore, the size of the image capturing areas is determined by the field corresponding to the magnification of the microscope 1. The number of the image capturing areas is not limited to the example shown in FIG. 3. Each image capturing area AA through ZZ is observed in order by the measuring device.

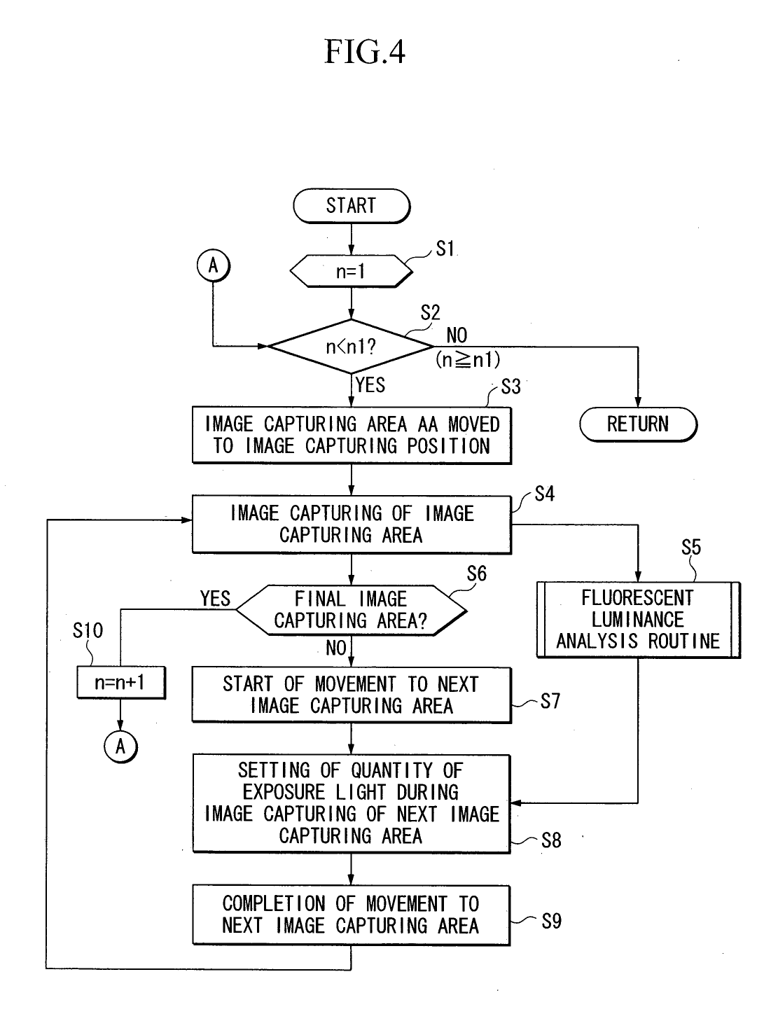

During the image capturing, the control device 2 controls the capturing of fluorescent images by executing a predetermined program. An explanation of the processing at this time is explained while referring to the flow charts shown in FIG. 4 through FIG. 6.

First, the control device 2 sets and confirms the number of times images are to be captured in the form of a preparation step. Namely, the value of the number of the image captures n is initially set to “1” (Step S1). In the initial image capture, since the number of the image captures n is less than the predetermined number of the image captures n1 (Yes in Step S2), the image capturing begins. It should be noted that the predetermined number of the image captures n1 is an integer of 2 or more.

During the start of the image capturing, the drive signal is output from the control device 2 to the motorized stage 11 and the image capturing area AA is moved to within the field of view of the objective lens 29 (Step S3).

Once the movement of motorized stage 11 is completed, the fluorescent image in the image capturing area AA is captured (Step S4). More specifically, the excitation light from the optical system 13 is radiated onto the image capturing area AA. The excitation light is obtained by passing light from the light source 12 through the excitation filter 27. The shape of the excitation light is adjusted by the lenses 23, 26 and 29 and by the stops 24 and 25 to match the shape of the image capturing area AA.

When irradiated with this excitation light, fluorescence attributed to a fluorescent protein in the form of GFP is generated in the sample. This fluorescent light passes through the objective lens 29 and the dichroic mirror 28. After surplus wavelength components have been removed with the excitation light cutoff filter 14, the fluorescent light is reflected by the mirror 15 toward the imaging lens 16. It is then guided to the imaging lens 16 where it focuses an image in the light receiving, section of the CCD camera 18. At this time, the motorized variable ND filter 31 having the previously described constitution is arranged between the imaging lens 16 and the CCD camera 18. Thus, the quantity of the excitation light that forms the image in the CCD camera 18 is the quantity of light corresponding to the density of the ND filter 31 in the light path. In addition, the quantity of the exposure light can be controlled using the electronic shutter of the CCD camera 18.

Once the fluorescent image of the image capturing area AA has finished being captured, the control device 2 incorporates and stores the fluorescent image in the terminal device 3. Then, an analysis is carried out according to the fluorescent intensity analysis routine based on this fluorescent image (Step S5). The details of the fluorescent intensity analysis routine (FIG. 6) will be described later.

Once the fluorescent image of the image capturing area AA has finished being captured, the control device 2 judges whether or not the captured area is the final image capturing area of the sample (Step S6). Here, since the image capture of the first observation area AA has just been completed (No in Step S6), the motorized stage 11 begins to move so as to move the next image capturing area in the form of the image capturing area AB to the field of view of the objective lens 29 (Step S7). At this time, the positions of the CCD camera 18 and other constituents remain fixed.

The motorized stage 11 should be moved in the direction of the X axis in FIG. 3 in order to move the image capturing area AB to the irradiated location. During this time, the control device 2 sets the quantity of image capturing exposure light for the image capturing area AB (Step S8). When the number of the image captures n is 1, namely during the first image capture, the quantity of the image capturing exposure light is not allowed to be changed for each image capturing area.

Once the image capturing area AB has moved to the irradiated location (Step S9), operation returns to Step S4 and a fluorescent image of the image capturing area AB is captured.

Subsequently, in FIG. 3, the motorized stage 11 is moved one image capturing area at a time in the direction of the X axis from the last image capturing area in the direction of the X axis to the image capturing area AZ to sequentially acquire fluorescent images. When image capturing of the image capturing area AZ has been completed, the motorized stage 11 is moved in the negative direction in the direction of the X axis and returned to the initial location. It is then moved in the positive direction one image capturing area at a time in the direction of the Y axis. As a result, images are captured for the line of the image capturing areas BA through BZ continuing from the image capturing areas AA through AZ.

Once the image capturing has been completed for the final image capturing area in the form of the image capturing area ZZ, operation proceeds from Step S6 to Step S10 and the number of the image captures n is incremented by one and set to “2”. Subsequently, when carrying out the second round of image capturing and beyond, the aforementioned processing is repeated at a predetermined timing so that each image capturing area AA through ZZ is observed over time. More specifically, after having set the number of image captures n to “2” in Step S6, operation proceeds to Step S2 from terminal A to judge the number of image captures. In the case the predetermined number of the image captures n1 is greater than 2, processing is repeated starting from Step S3. The image capturing ends when the second round of image capturing and beyond is carried out and the number of the image captures n becomes equal to the predetermined number of the image captures n1 (No in Step S2).

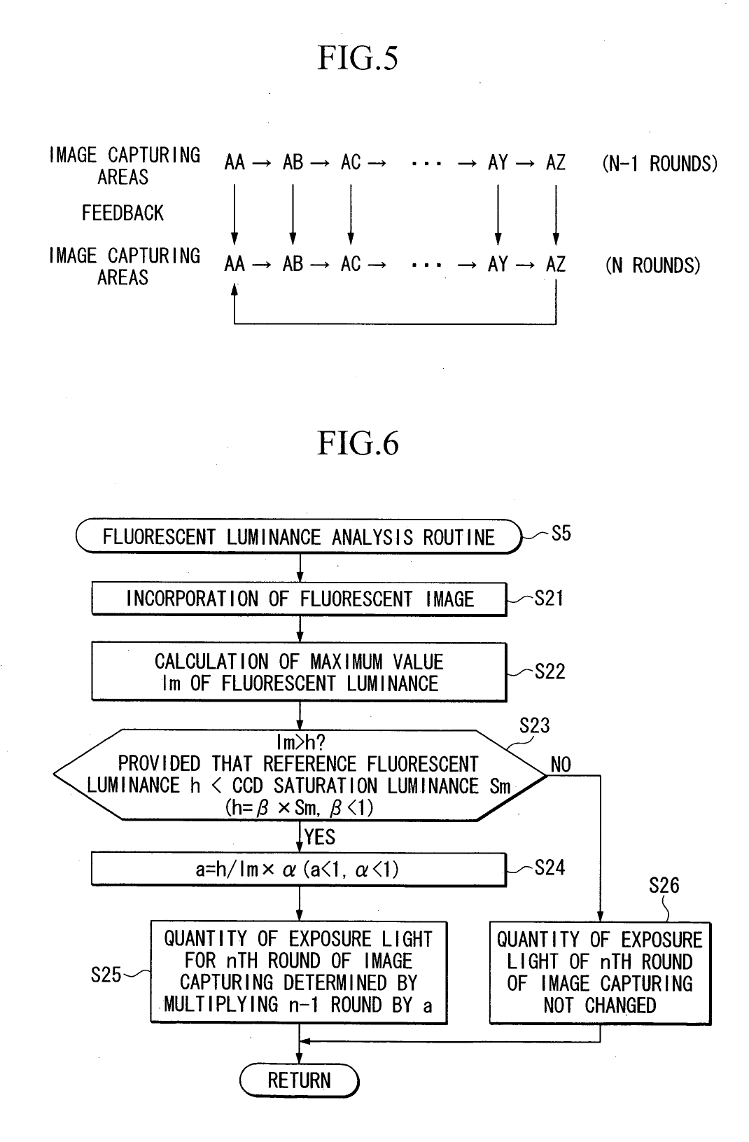

Since the amount of the tissue and so forth that emits fluorescence varies for each image capturing area AA through ZZ, the quantity of the fluorescent light received by the CCD camera 18 also varies for each image capturing area AA through ZZ. Consequently, during the second round of image capturing and beyond, if all image capturing areas AA through ZZ are captured with the same quantity of the exposure light, there is the possibility of fluorescent luminance becoming saturated or fluorescent luminance being so weak that it cannot be detected. Thus, in the second round of the image capturing and beyond, the image capturing is carried out after changing the quantity of exposure light used during the image capturing for each image capturing area AA through ZZ in Step S8. In other words, as shown in FIG. 5, in the nth round of the image capturing, information on fluorescent luminance is fed back to the n-1 round. The electronic shutter of the CCD camera 18 or the rotated position of the motorized variable ND filter 17 as shown in FIG. 2 is set as necessary to adjust the quantity of exposure light during the image capturing.

The following provides an explanation of the details of the fluorescent luminance analysis routine (Step S5 in FIG. 4) that carries out processing for adjusting the quantity of exposure light during the image capturing with reference to FIG. 6.

First, the control device 2 incorporates the fluorescent image (Step S21). The maximum value Im of fluorescent intensity is then calculated from the image data of the fluorescent image (Step S22). Next, a comparison is made of the magnitude of the maximum value lm and a predetermined reference fluorescent luminance h (Step S23). Furthermore, a reference fluorescent luminance h is the quantity resulting from multiplying a coefficient β (β<1) by the luminance at which the CCD camera 18 is saturated (CCD saturation luminance Sm). The CCD saturation luminance Sm is a constant determined by the CCD. The coefficient β is a parameter that determines to what extent the dynamic range of the CCD camera 18 is used. For example, if β=0.9, then the CCD camera 18 is used up to 90% of the maximum measurable luminance without becoming saturated.

The reference fluorescent luminance h is considered to have the possibility of the fluorescent luminance becoming saturated during the next image capture if lm exceeds h (Yes in Step S23), and coefficient a is then calculated so as to reduce the next quantity of fluorescent exposure light (Step S24). The coefficient a is the value obtained by multiplying the value obtained by dividing the reference fluorescent luminance h by the maximum value lm times α (α<1). α is a weighting parameter that determines to what extent the next quantity of fluorescent exposure light should be suppressed. For example, α should be set to a small value for an image capturing area having large fluctuations in the fluorescent luminance, and should be set to a large value for an image capturing area having small fluctuations. The coefficient a is always less than 1.

The quantity of the exposure light for the n-th round of the image capturing is set to a value obtained by multiplying the quantity of the exposure light for the n-1 round of the image capturing by the coefficient a. Subsequently, this routine is ended and the operation returns to Step S8 in FIG. 4.

If the maximum value lm is less than the CCD saturation luminance h (No in Step S23), the quantity of the exposure light for the next round of the image capturing is not changed (Step S26), and the operation returns to Step S8 in FIG. 4.

According to this embodiment, the quantity of the exposure light for the subsequent image capturing is adjusted based on the maximum value of the fluorescent luminance during the previous image capturing at the same location using the fluorescent luminance analysis routine. Namely, the quantity of the exposure light for the fluorescent image subsequently acquired is adjusted based on the information obtained from the previously acquired fluorescent image at the same image capturing area. Accordingly, even if the fluorescent intensity of the cells increases during the observation, the fluorescent luminance does not become saturated when observing the cells or other objects being observed.

In addition, there are limitations on the operation speed of the electronic shutter. There is a case, therefore, in which the intensity of the fluorescent light input to the image capturing system is so large that the fluorescent intensity becomes saturated even when using the maximum operation speed (video rate) of the shutter to reduce the quantity of the exposure light as much as possible. Even in the case, the saturation of fluorescent intensity can be prevented by rotating the motorized variable ND filter 17 in order to decrease the transmittance of the fluorescent light.

Moreover, in the case of detecting extremely weak fluorescent light, the fluorescent images can be captured by increasing the amount of the exposure light with the electronic shutter while scanning the object being observed a plurality of times. Therefore, well-defined fluorescent images can be obtained without being affected by amplified noise, which tends to occur in the manner of creating fluorescent images by electrically amplifying the electrical image signal. This is the result of the signal level generated by the light receiving section of the CCD camera 18 being positively proportional to the exposure time. Namely, this is because if all other conditions are equal, the signal level becomes larger the longer the exposure time, and when the signal level becomes larger, the signal-to-noise ratio (SN ratio) between pixels improves relative to the case of short exposure time.

Moreover, since the fluorescence can be observed over a range that matches the fluorescent luminance at the portion being observed, it is possible to take advantage of the dynamic range of the image capturing system. As a result of these effects, the images of the fluorescent intensity generated from the body tissue can be acquired accurately. In addition, since the measuring conditions can be determined on a real-time basis without requiring preliminary scanning, the amount of time required to measure the entire sample can be reduced.

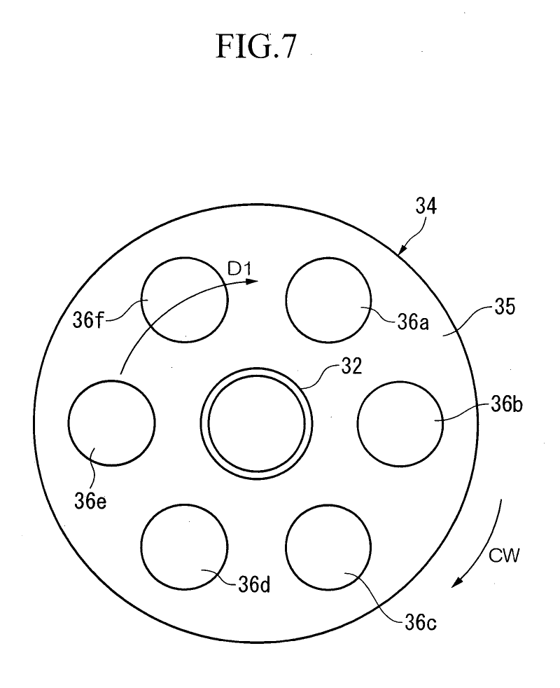

FIG. 7 shows an another example of a motorized variable ND filter. The motorized variable ND filter 34 shown in FIG. 7 has a plurality of ND filters 36a through 36f along circumferential direction D1 on a disk 35 fixed to a rotation axis 32. The ND filters 36a through 36f have transmittance values that vary in a stepwise manner, respectively. The rotation axis 32 is rotated and controlled so that any of the ND filters 36a through 36f is arranged in the light path. Thus, the transmittance of the filter is increased or decreased in a stepwise manner when the rotation axis 32 is rotated in the clockwise direction.

According to this type of motorized variable ND filter 34, the quantity of the exposure light can be changed to a suitable value by choosing one of the ND filters 36a through 36f in response to the quantity of the exposure light for the image capturing obtained by the fluorescent luminance analysis route (FIG. 6).

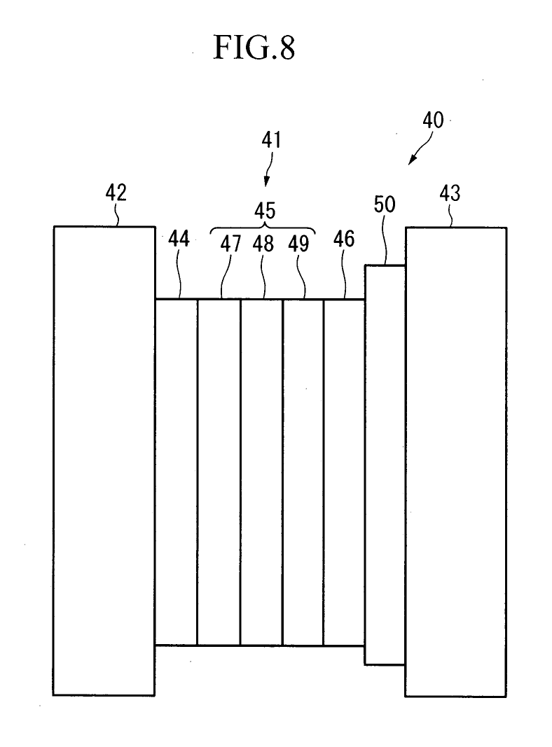

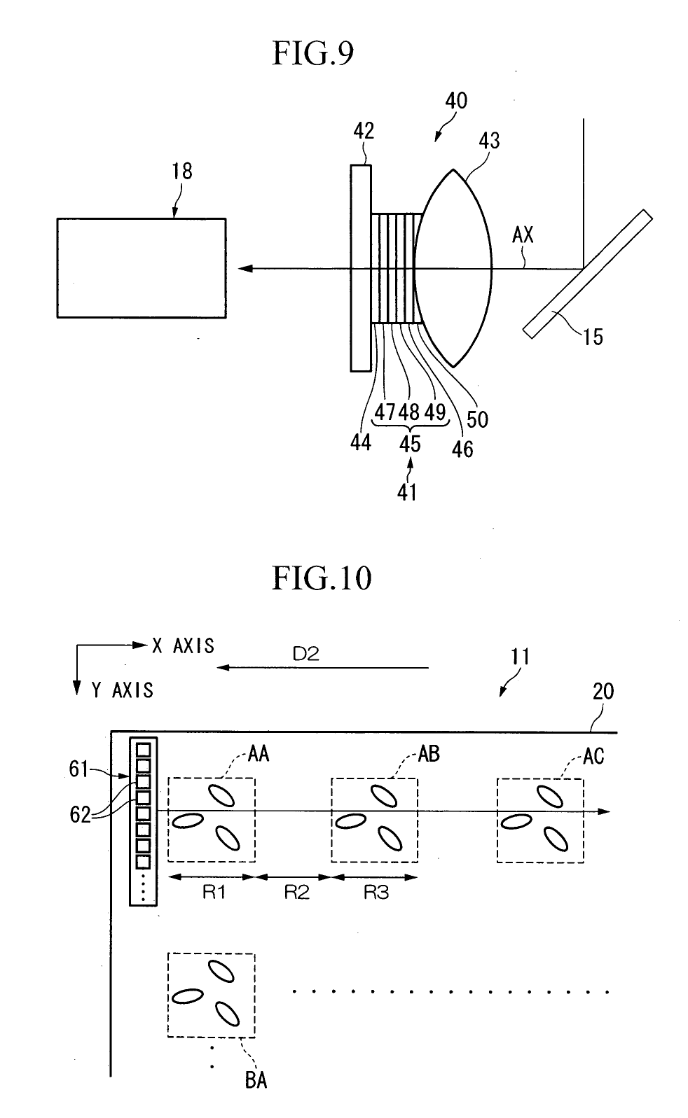

Next, an explanation is provided of a measuring device according to a second embodiment of the present invention with reference to FIGS. 8 and 9. In the drawings, those constituents that are the same as those in the first embodiment are indicated with the same reference symbols.

In this second embodiment, an electrochromic light adjusting device is used for the means of changing the quantity of exposure light for image capturing. This electrochromic light adjusting device is arranged between the mirror 15 and the CCD camera 18 in the measuring device of the first embodiment. Other constituents are the same as in the first embodiment shown in FIG. 1, and their explanations are omitted to avoid repetition. As known in the art, the electrochromic light adjusting device refers to a device using a substance that changes the transmittance of light by a chemical change in response to a quantity of electricity.

As schematically shown in FIG. 8, an electrochromic light adjusting device 40 has a constitution in which an electrochromic layer 41 is interposed between two support layers 42 and 43. The support layers 42 and 43 are produced from a material that is transparent to the fluorescent light. The electrochromic layer 41 is an electrical light adjusting device in which a transparent electrically conductive film 44, an electrochemical variable transmittance substance 45 and a transparent electrically conductive film 46 are laminated in that order from the side of the support layer 42. An indium tin oxide (ITO) film, for example, is used for the transparent electrically conductive films 44 and 46. The variable transmittance substance layer 45 has a three-layer structure. A first film 47 composed of tungsten trioxide (WO₃), for example, is formed on the transparent electrically conductive film 44. A second film 48 is formed on this first film 47. The second film 48 is composed of, for example, dithallium pentoxide (T₂O₅). A third film 49 is formed on the second film 48. This third film 49 is composed of nickel oxide (NiO), for example. Each film 44, 46 and 47 through 49 is formed by coating or sputtering in order on the support layer 42. The transparent electrically conductive film 46 and the support layer 43 are adhered with an adhesive 50.

This type of electrochromic light adjusting device 40 is arranged closer to the support 20 than the CCD camera 18 as shown in FIG. 1 in place of the imaging lens 16 and the motorized variable ND filter 17. Each electrically conductive film 44 and 46 is connected to a control unit in the form of the control device 2.

An example of the arrangement of the electrochromic light adjusting device 40 is shown in FIG. 9. Here, the support layer 43 serves as an imaging lens, and the electrochromic light adjusting device 40 and the imaging lens are integrally composed. This imaging lens is designed so that the fluorescent light forms an image in the light receiving section of the CCD camera 18. A parallel flat plate that is perpendicular to optical axis AX of the fluorescent light is used for the support layer 42.

In the same manner as the first embodiment, the control device 2 captures the fluorescent images of an observed object in accordance with a flow chart like that shown in FIGS. 4 and 6. At this time, in the processing equivalent to Step S8 in FIG. 4, the control device 2 adjusts the transmittance of the electrochromic layer 41 by changing the voltage applied between the electrically conductive films 44 and 46 of the electrochromic light adjusting device 40 in response to the quantity of the exposure light for the image capturing. As a result, the quantity of the fluorescent light that enters the CCD camera 18 is adjusted to the quantity of exposure light set with the fluorescent luminance analysis routine shown in FIG. 6.

According to this second embodiment, effects are obtained that are the same as those obtained in the first embodiment. Moreover, as a result of using the electrochromic light adjusting device 40, the transmittance of the fluorescent light can be adjusted based on the voltage value applied to the transparent electrically conductive films. The quantity of the exposure light for the image capturing can, therefore, be adjusted accurately and in a short period of time.

Thus, even in cases in which the intensity of the fluorescent light that enters the image capturing element is so large that fluorescent intensity becomes saturated even when the quantity of the exposure light is reduced as much as possible, the fluorescent intensity no longer becomes saturated by changing the optical transmittance of the electrochromic light adjusting device under the electrical control.

Moreover, the following provides an explanation of another example of a means of changing the quantity of the exposure light for the image capturing.

A mechanical shutter may be used instead of the electronic shutter of the CCD camera 18. In this case, a shutter plate is provided in the light path between the imaging lens 16 and the CCD camera 18 shown in FIG. 1. This shutter is opened and closed in response to a control signal from the control device 2.

In this case, the time during which the shutter is closed, namely the time during which the shutter blocks the light path, is changed based on the processing of the fluorescent luminance analysis routine shown in FIG. 6. As a result, the quantity of the exposure light for the image capturing can be changed to a suitable quantity. Thus, the same effects as those obtained in the first embodiment can be obtained.

Namely, the quantity of the exposure light for the image capturing can be changed to a suitable quantity by changing the rate of opening and closing of the mechanical shutter. Therefore, the fluorescent intensity no longer becomes saturated. In addition, in the case of detecting extremely weak fluorescent light, the quantity of the exposure light for the image capturing unit is changed instead of electrically amplifying an image signal obtained by image capturing. Accordingly, the noise in the fluorescent image is not amplified.

Next, an explanation is provided of a measuring device according to a third embodiment of the present invention with reference to FIG. 10. In the drawing, those constituents that are the same as those in each of the aforementioned embodiments are indicated with the same reference symbols, and their explanations are omitted to avoid repetition.

Although an area type CCD was used for the image capturing unit in the first and second embodiments, in this third embodiment, a line (or liner) CCD camera that uses a linear type of CCD is used for the image capturing unit. The quantity of the exposure light for image capturing is adjusted by controlling the video rate of the line CCD camera and the scanning speed of the motorized stage.

As shown in FIG. 10, line CCD camera 61 has sensors in the form of CCD 62 arranged in a row in the direction of the Y axis, and is fixed in coordinate plane XY In this measuring device, the control device 2 as shown in FIG. 1 moves the motorized stage 11 in the negative direction (refer to arrow D2) along the X axis. As a result, the support 20 and the sample are relatively scanned in the positive direction of the X axis by the line CCD camera 61.

Here, the arrangement pitch, namely the minimum pixel pitch, of each CCD 62 is defined as p (mm). The video rate is defined as Lt (Hz). The total microscopic magnification at the surface of the CCD 62 relative to the sample surface on the motorized stage 11 is defined as M. Since the video rate is equivalent to the amount of time taken to read out one line of the CCDs 62, the quantity of the exposure light for image capturing at that time becomes 1/Lt (sec). Accordingly, by changing the video rate Lt of the line CCD camera 61 according to the maximum value of the fluorescent luminance during the previous image capture, the quantity of the exposure light during the next image capture in the same image capturing area can be adjusted to a suitable quantity.

If the stage moving speed of the motorized stage 11 is made to be (p/M)×Lt (mm/sec), the stage speed can be synchronized to the video rate of the line CCD camera 61. Thus, signals based on the fluorescent light can be read accurately.

In the case it is desired to change the quantity of the exposure light for the image capturing between the image capturing area AA and the image capturing area AB, then the stage speed should be changed between the zone corresponding the image capturing area AA indicated with R1 in the drawing and the zone corresponding to the image capturing area AB indicated with R3. Switching of the stage speed is carried out during the movement in a zone R2 located between the zone R1 and the zone R3.

In FIG. 10, in the case of capturing a fluorescent image in the image capturing area BA, for example, the image capturing is carried out after putting the line CCD camera 61 in the non-operating state and moving the motorized stage 11 in the positive direction along the Y axis.

According to this third embodiment, the same effects as those of the first embodiment can be obtained by composing a unit that changes the quantity of the exposure light for the image capturing with the line CCD camera 61 and the motorized stage 11. In particular, since the quantity of the exposure light for the image capturing can be adjusted by adjusting the stage speed of the motorized stage 11, the quantity of the exposure light can be controlled reliably and the fluorescent intensity no longer becomes saturated. Thus, noise-free and clear fluorescent images are obtained. Moreover, the constitution of the image capturing system can be simplified. In addition, the time required for the image capturing can be reduced since multiple scans of the sample are no longer required.

In addition, in the case of detecting extremely weak fluorescent light, the quantity of the exposure light for the image capturing unit can be changed instead of electrically amplifying an electric image signal obtained by image capturing. The noise in the fluorescent image is, therefore, not amplified.

Next, an explanation is provided of a measuring device according to a fourth embodiment of the present invention with reference to FIG. 1. In the drawing, those constituents that are the same as those in each of the aforementioned embodiments are indicated with the same reference symbols, and their explanations are omitted to avoid repetition.

The fourth embodiment uses a time delay integration (TDI) type of line (linear) CCD camera for the image capturing unit, and changes the quantity of the exposure light for the image capturing according to the number of stages of the TDI line of the TDI type of line CCD camera.

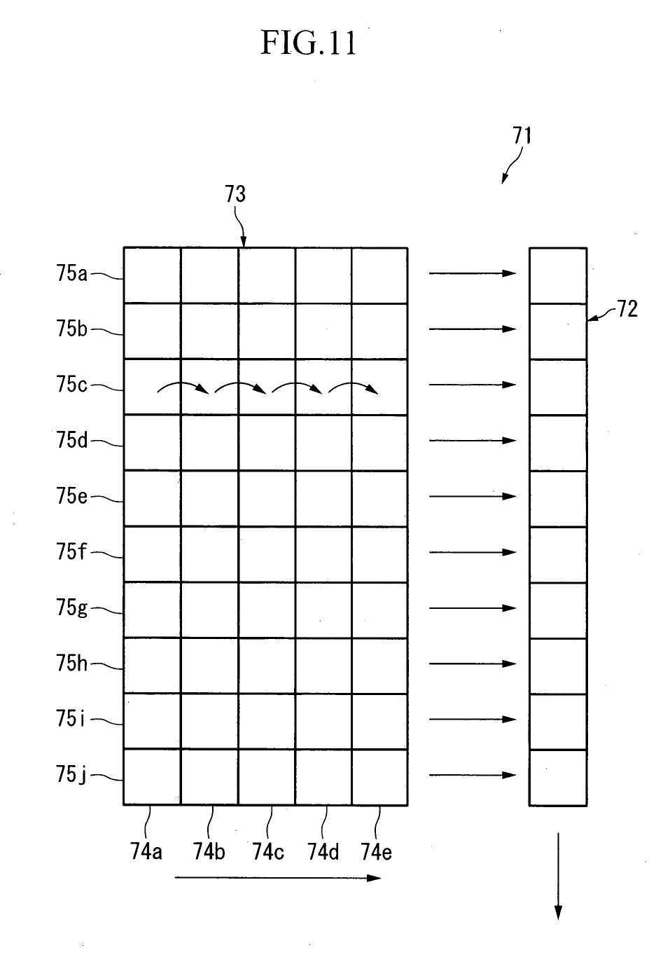

As shown in FIG. 11, a TDI type of line CCD camera 71 includes a shift register 72, TDI detector 73 and an amplifier (not shown). The TDI detector 73 has N number of stages (number of stages N=5 in FIG. 11) 74a through 74e. Each stage 74a through 74e has a plurality of columns 75a through 75j.

In the TDI type of the line CCD camera 71, an image obtained from a scanning sensor not shown is input to the TDI detector 73. In the TDI detector 73, the image moves from the stage 74a located farthest to the left to the stage 74e located farthest to the right in FIG. 11. Since the electrical charge also moves roughly synchronous with the image, a total electrical charge equal to five times that in the case of single linear detection is generated at the point the image has passed through all five stages of the TDI detector 73.

Moreover, the total sum of the electrical charge is transmitted to the shift register 72. This electrical charge is then output to the amplifier during the time the image charge of the next line reaches the shift register 72 from the TDI detector 73. Furthermore, since this overall process is in the form of a pipeline, a portion of the image is continuously emitted to each pixel.

When the video rate of the TDI type of the line CCD camera 71 is taken to be Lt (Hz) and the number of stages of the TDI line is taken to be N, then the quantity of the exposure light for the image capturing becomes (1/Lt)×N (sec). Accordingly, the quantity of the exposure light for the image capturing can be adjusted to the optimum quantity by changing the number of stages N of the TDI type of the line CCD camera according to the maximum value of the fluorescent luminance during the previous image capture.

According to the fourth embodiment, the same effects as the first embodiment can be obtained by using the TDI type of the line CCD camera 71 as a means of changing the quantity of the exposure light for the image capturing. In particular, image capturing time can be shortened since multiple scans of the sample are no longer required.

Moreover, according to this measuring device, the fluorescent intensity is no longer saturated since the quantity of the exposure light for the image capturing can be changed to a suitable quantity by changing the number of stages of the line of the time delay integration type of the line solid-stage image capturing unit while synchronizing the video rate of the time delay integration type of solid-stage line image capturing unit with the operating speed of the motorized stage. In addition, in the case of detecting weak fluorescent light, the noise in the fluorescent images is not amplified since the quantity of exposure light of the image capturing unit is changed instead of electrically amplifying an electrical signal obtained by image capturing.

The maximum number of stages of the TDI type of the line CCD camera 71 is not limited to five, but rather may be any arbitrary integer. In addition, the number of columns is not limited to 10 as shown in FIG. 11.

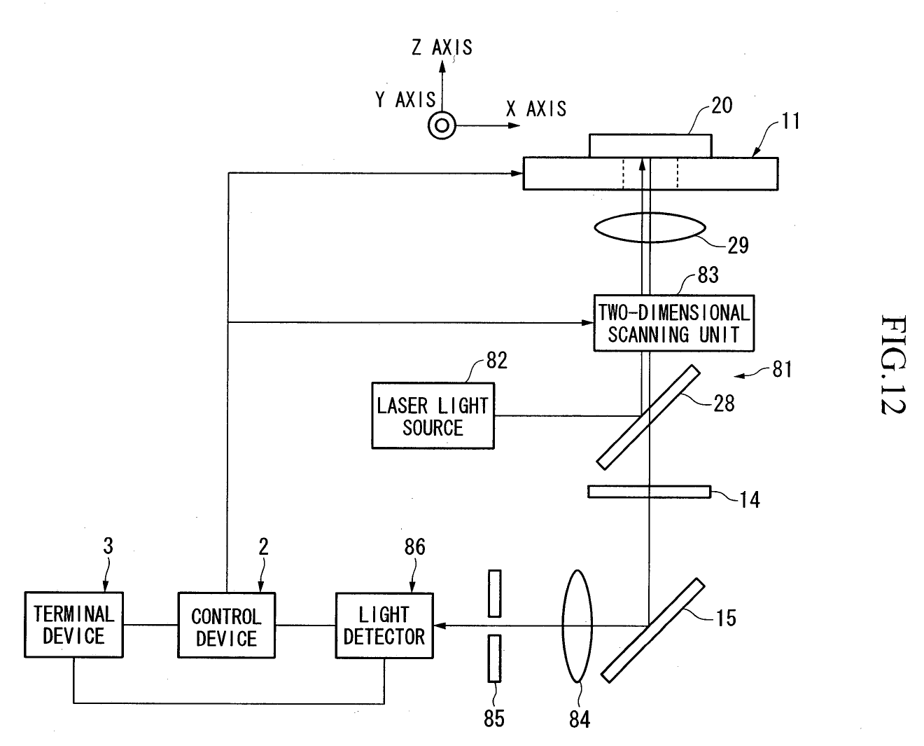

Next, an explanation of a measuring device according to a fifth embodiment of the present invention is provided with reference to FIG. 12. In the drawing, those constituents that are the same as those in each of the aforementioned embodiments are indicated with the same reference symbols, and their explanations are omitted to avoid repetition.

In the fifth embodiment, a laser scanning microscope is used for the microscope. Moreover, the quantity of the exposure light for the image capturing is adjusted by controlling the scanning speed of the scanning mirror of the laser scanning microscope.

As shown in FIG. 12, the measuring device comprises a laser scanning microscope 81, the control device 2 and the terminal device 3.

The laser scanning microscope 81 contains a laser light source 82, a dichroic mirror 28, a two-dimensional scanning unit 83, an objective lens 29, a motorized stage 11, an excitation light cutoff filter 14, a mirror 15, a lens 84, a pinhole 85 and a light detector 86. A laser light source that emits laser light of a wavelength corresponding to the excitation light is used for the laser light source 82. The two-dimensional scanning unit 83 is interposed between the dichroic mirror 28 and the objective lens 29. This constitution contains a galvanometer mirror that scans laser light in the direction of the X axis and a galvanometer mirror that scans laser light in the direction of the Y axis.

The lens 84 is arranged in the light path of the fluorescent light reflected with the mirror 15. The pinhole 85 is arranged at the location where light from the lens 84 converges, and forms a confocal optical system. The light detector 86 outputs an electrical signal as a result of entry of the fluorescent light.

The control device 2 is connected to the motorized stage 11, the two-dimensional scanning unit 83, the light detector 86 and the terminal device 3. The terminal device 3 is connected to the control device 2 and the light detector 86.

In the measuring device, excitation light from the laser light source 82 is reflected towards a sample by the dichroic mirror 28 and is guided to the two-dimensional scanning unit 83. The two-dimensional scanning unit 83 then scans the two galvanometer mirrors based on a scanning control signal from the control device 2.

As a result, the scanning unit 83 scans the excitation light along the sample in both the directions of the X and Y axes. A scanning completion signal is output to the control device 2 each time the two-dimensional scanning unit 83 completes scanning of the excitation light in the direction of the X axis. Since the excitation light is converged by the objective lens 29, it is irradiated onto the sample in the form of light spot.

Image information in the form of the fluorescent light is generated from the sample as a result of irradiation with the excitation light. The fluorescent light returns in the opposite direction along the light path by which the excitation light entered and passes through the dichroic mirror 28. Moreover, after passing through the excitation light cutoff filter 14, it is reflected by the mirror 15. The fluorescent light is then converged by the lens 84, passes through the pinhole 85 and enters the light detector 86. An electrical signal corresponding to the quantity of the fluorescent light is output in the light detector 86. This electrical signal is then input to the control device 2. Moreover, the electrical signal from the light detector 86 is also input to the terminal device 3 where it is converted to an image in the form of the fluorescent image of the sample. Since the laser scanning microscope 81 forms the confocal optical system, the fluorescent image has a cross-sectional image of the sample in the XY plane that is perpendicular to the direction of the Z axis.

When adjusting the quantity of the exposure light for the image capturing, the amount of the exposure light for the second round of the image capturing and beyond is set for each image capturing area using the fluorescent luminance analysis routine like that shown in FIG. 6. The control device 2 then changes the scanning speed of the laser light using the galvanometer mirrors located within the two-dimensional scanning unit 83 based on the set quantity of the exposure light. Namely, the quantity of the exposure light for the image capturing becomes smaller the faster the scanning speed, while the quantity of exposure light for image capturing becomes larger the slower the scanning speed.

According to the fifth embodiment, the direction of cell excitation is changed and the quantity of the exposure light when capturing the fluorescent image is adjusted by controlling the scanning speed of the excitation light in the form of laser light using the laser scanning microscope 81. The saturation of the fluorescent luminance when observing cells and other observed objects can therefore be prevented. In addition, it is also possible to take advantage of the dynamic range of the light detector 86.

Thus, images of the fluorescent intensity generated from body tissue and so forth can be acquired accurately. In addition, preliminary scanning is not required. The time required to measure an entire sample can be shortened since measuring conditions can be determined on a real-time basis.

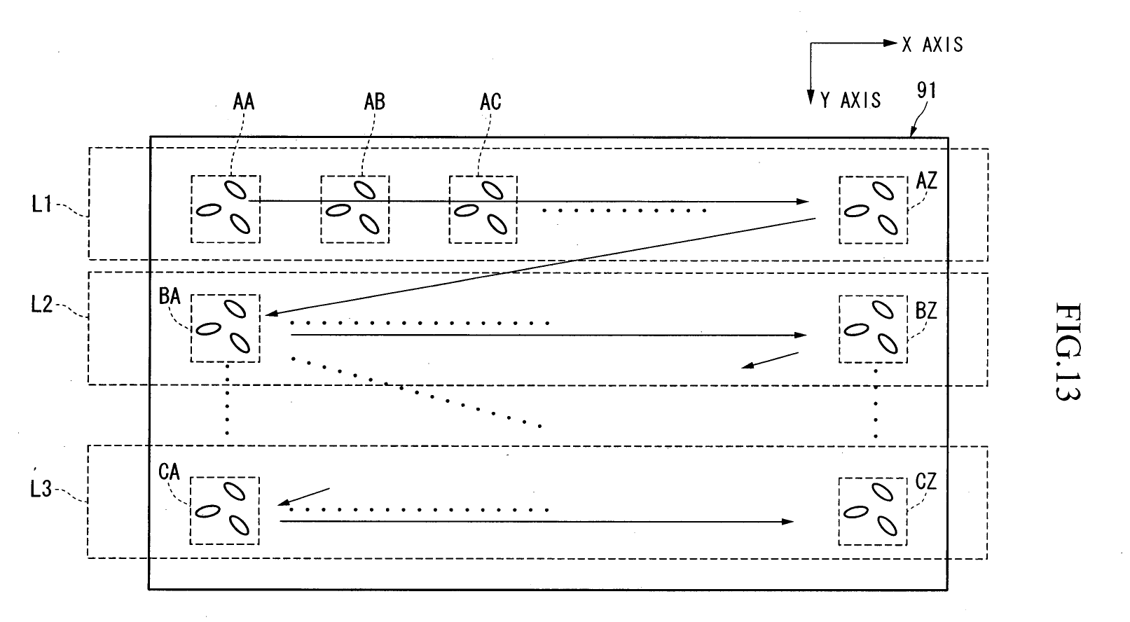

Next, an explanation is provided of the case of measuring a sample composed of a plurality of cells as shown in FIG. 13 using a measuring device according to the aforementioned first through fifth embodiments.

Each cell is arranged in the form of a grid on a support 91 as shown in FIG. 13. The support 91 is composed of a slide glass, and has a constitution in which a plurality of line areas L1, L2 and L3 are arranged along the direction of the X axis. A plurality of cells are retained in each line area L1 through L3 at a predetermined distance. Image capturing areas AA through CZ shown here correspond to each cell arranged on the support 91.

In this case, fluorescent images are captured of the cells arranged in the form of a grid by scanning in order, and the quantity of the exposure light for capturing images is adjusted for each cell. The use of this type of the support 91 makes it possible to observe the fluorescence of a plurality of cells in order.

In this type of the support 91, the cells can be arranged and classified using each line area L1 through L3. More specifically, the cells are arranged in each line area L1 through L3 that have similar magnitudes of fluorescent luminance when a gene is expressed. The quantity of the exposure light for the image capturing is then set for each line area L1 through L3 using the changing means in each of the aforementioned embodiments (for example, the motorized variable ND filter 17 shown in FIG. 1).

For example, a group of the cells having a comparatively large fluorescent luminance is arranged in line area L1 while a group of the cells having a relatively lower fluorescent intensity than the line area L1 is arranged in line area L2.

The quantity of the exposure light for the image capturing is set to a low level in the line area L1 using a means of regulating the quantity of the exposure light described in each of the aforementioned embodiments (for example, the motorized variable ND filter 17 shown in FIG. 1). Fluorescent images of each cell are then captured while maintaining the quantity of the exposure light to a predetermined value in the line area L1. When capturing images of cells in the line area L2, the quantity of the exposure light for the image capturing is increased to a level higher than that of the line area L1. The quantity of the exposure light for the image capturing in this line area L2 is then maintained at a constant level.

Whereupon, it is not necessary to change the quantity of the exposure light for the image capturing for each grid, namely for each cell, provided the cells are within the same line areas L1 through L3. Adjustment of the quantity of the exposure light is only required to be carried out when changing the line area. Thus, the number of times of operating the means for changing the quantity of the exposure light for the image capturing can be reduced. Moreover, since the time for operating the means for changing the quantity of the exposure light can also be reduced accompanying this, the entire support 91 can be scanned at high speed. Thus, fluorescent images can be captured rapidly.

When capturing images in the next line area after having captured images in the previous line area, for example, when moving from the image capturing area AZ to the image capturing area BA in FIG. 13, the quantity of the exposure light for the image capturing is adjusted with a changing means like that explained in each of the aforementioned embodiments during time of moving between the line areas. In this case, since the quantity of the exposure light for the image capturing is adjusted while moving the motorized stage 11, the entire support 91 can be scanned efficiently.

In each of the aforementioned embodiments, the control device 2 and the terminal device 3 may be integrated into a single device. In addition, in each of the embodiments, a program that executes processing like that shown in FIGS. 4 and 6 in a computer, and a recording medium on which this program is recorded so as to be able to be accessed from a computer and so forth are also included in embodiments of this invention.

According to the present invention, the fluorescent observations are made in a range that matches the fluorescent luminance at a site irradiated by the excitation light. The saturation of the fluorescent luminance during measurement can therefore be prevented while taking advantage of the dynamic range of the image capturing system. In addition, since the fluorescent luminance is not increased or decreased electrically, the noise in the fluorescent image can be reduced. As a result, information on the fluorescent intensity generated from body tissue can be acquired accurately. Moreover, since the image capturing conditions can be determined on a real-time basis without requiring preliminary scanning, the time required to measure the sample can be shortened.

While the preferred embodiments of the invention have been described and illustrated above, it should be understood that these are exemplary of the invention and are not to be considered as limiting. Additions, omissions, substitutions and other modifications can be made without departing from the spirit or scope of the present invention. Accordingly, the invention is not to be considered as being limited by the foregoing description, and is only limited by the scope of the appended claims.

1. A fluorescence observation method comprising the steps of: arranging cells to be observed on a support; acquiring images of fluorescence emitted from said cells as a result of irradiating with excitation light at predetermined time intervals with a time-based image capturing system; and determining quantity of exposure light for capturing a fluorescent image to be subsequently acquired using a previously acquired fluorescent image.

2. A measuring device comprising:
a motorized stage that moves a support on which cells to be observed are arranged;
a light source used to radiate excitation light onto said cells;
an objective lens that converges said excitation light from said light source towards said cells;
an excitation light cutoff filter that cuts out light of a predetermined wavelength from return light reflected from said support;
an imaging lens that forms an image of a fluorescent light emitted from said cells on said support at a predetermined image capturing position;
an image capturing device arranged at said image capturing position; and
a processing device that incorporates fluorescent images captured by said image capturing device and performs image processing on said fluorescent images; wherein,
said processing device sets an image capturing conditions of said fluorescent image to be acquired next based on image processing results of the previously acquired fluorescent image.

3. The measuring device according to claim 2 wherein, said image capturing device is a solid-state image processing device provided with photoelectric conversion elements and an electronic shutter.

4. The measuring device according to claim 2 wherein, a mechanical shutter is provided in said image capturing device, said mechanical shutter being driven based on said image capturing conditions.

5. The measuring device according to claim 2 wherein, said image capturing device is a line solid-state image capturing device in which photoelectric conversion elements are arranged in the form of a line, and a scanning speed of said line solid-state image capturing device is changed while synchronizing a video rate of said line solid-state image capturing device and a scanning speed of said motorized stage based on said image capturing conditions.

6. The measuring device according to claim 2 wherein, said image capturing device is a time delay integration type of line solid-state image capturing device, and the number of stages of said time delay integration type of line solid-state image capturing unit is changed while synchronizing a video rate of said time delay integration type of line solid-state image capturing unit and a scanning speed of said motorized stage based on said image capturing conditions.

7. The measuring device according to claim 2 wherein, said measuring device includes a laser light source for said light source and a scanning unit that scans laser light from said laser light source, a scanning speed of said laser light by said scanning unit being changed based on said image capturing conditions.

8. The measuring device according to claim 2, further comprising a neutral density filter provided upstream from said fluorescent light that enters said image capturing device, and a drive unit driving said neutral density filter, said neutral density filter being driven based on said image capturing conditions.

9. The measuring device according to claim 2, further comprising an electrochromic light adjusting device provided upstream from said fluorescent light that enters said image capturing device, and a control unit controlling said electrochromic light adjusting device, said electrochromic light adjusting device being controlled based on said image capturing conditions.

10. The measuring device according to claim 2 wherein, said support is a slide glass, and said cells are arranged on said slide glass in the form of a grid.

11. The measuring device according to claim 10 wherein, a plurality of said cells having a similar magnitude of fluorescent intensity when a gene has been expressed are arranged in rows on said slide glass along a direction of movement of said motorized stage, and said image capturing conditions are set for each row.

12. A measuring system for measuring changes in a cell comprising:
an image capturing device having: a support on which said cell to be observed is arranged; a light source that radiates excitation light onto said cell; an objective lens that converges said excitation light from said light source towards said cell; an imaging lens that focuses on an image capturing position a reflected light reflected from said cell as a result of being irradiated with said excitation light; and a photoelectric conversion sensor arranged at said image capturing position that converts said reflected light focused on said capturing position into an image of said cell; said image capturing device capturing first and second images of said cell under first and second optical conditions, respectively, and
a control device that analyzes said first image of said cell captured under said first optical condition from said image capturing device, determines said second optical condition in response to a results of analysis of said first image of said cell, and enables said image capturing device to capture said second image of said cell for which a predetermined period of time has elapsed under said second optical condition.

13. The measuring system according to claim 12 wherein, said first and second optical conditions define the first and second quantities of exposure of said reflected light that enters said photoelectric conversion sensor.

14. The measuring system according to claim 13 wherein, said image capturing device further has an adjuster for adjusting quantity of said reflected light that moves towards said photoelectric conversion sensor.

15. The measuring system according to claim 14 wherein, said adjuster includes a neutral density filter whose transmittance changes corresponding to a rotated position, and a rotator that drives said filter.

16. The measuring system according to claim 14 wherein, said adjuster includes an electrochromic light adjusting device whose transmittance changes corresponding to an applied voltage, and a controller that controls said voltage applied to said light adjusting device.

17. The measuring system according to claim 13 wherein, said image capturing device further has a laser light source as said light source, a scanning unit that scans laser light from said laser light source over said support, said scanning unit changing its scanning speed of said laser light in response to said first and second quantities of exposure.

18. The measuring system according to claim 13 wherein, said control device analyzes luminance of said first image of said cell captured by said first quantity of exposure, compares the analyzed luminance with a reference luminance, and determines said second quantity of exposure according to the results of the comparison of said analyzed luminance with said reference luminance.

19. The measuring system according to claim 18 wherein, said control device measures the maximum value of luminance of said first image of said cell captured by said first quantity of exposure, and compares said maximum value with said reference luminance determined based on a saturation luminance of said photoelectric conversion sensor.

20. The measuring system according to claim 19 wherein, said reference luminance is determined by multiplying said saturation luminance of said photoelectric conversion sensor by a coefficient β (β<1).

21. The measuring system according to claim 20 wherein, said coefficient β is 0.9.

22. The measuring system according to claim 19 wherein, said control device determines said second quantity of exposure by multiplying said first quantity of exposure by a coefficient a (a<1) when said maximum value of luminance of said first image of said cell is greater than said reference luminance.

23. The measuring system according to claim 22 wherein, said coefficient a is the product of multiplying the value obtained by dividing said reference luminance by said maximum value by α (α<1).

24. The measuring system according to claim 12 wherein, said image capturing device further has a moving stage that relatively moves said support relative to said objective lens, and a plurality of image capturing areas are defined on said support by the relative movement of said moving stage.

25. The measuring system according to claim 24 wherein, said first and second optical conditions are set for each of a plurality of said image capturing areas.

26. The measuring system according to claim 25 wherein, said control device controls said image capturing device so that the images of a plurality of said image capturing areas are captured under said second optical condition determined for each of a plurality of said image capturing areas after the images of a plurality of said image capturing areas have finished being captured under said first optical condition determined for each of a plurality of said image capturing areas by carrying out said relative movement of said moving stage.

27. The measuring system according to claim 24 wherein, a plurality of said image capturing areas are arranged on said support in the form of a grid.

28. The measuring system according to claim 27 wherein, said cells having a similar magnitude of fluorescent intensity when a gene has been expressed are arranged in a single direction in a plurality of said image capturing areas in the form of a grid.

29. The measuring system according to claim 12 wherein, said image capturing device further has an optical filter arranged in a light path of said reflected light, said optical filter extracting fluorescent light from said reflected light by cutting out a predetermined wavelength of said reflected light.

30. A method for measuring changes in a cell comprising the steps of:
radiating excitation light onto a support on which said cell to be observed is placed;
focusing on an image capturing position a reflected light reflected from said cell as a result of being irradiated with said the excitation light;
converting said reflected light focused on said image capturing position into an image of said cell;
obtaining a first image of said cell captured under a first optical condition;
determining a second optical condition based on an analysis of said first image of said cell; and
capturing a second image of said cell under said second optical condition when a predetermined period of time has elapsed after said first image has obtained.
